(19) United States
(12) Patent Application Publication
(10) Pub. No.: US 2024/0261044 A1
(43) Pub. Date: Aug. 8, 2024

(54) TOOL HEAD POSTURE ADJUSTMENT METHOD AND APPARATUS, AND READABLE STORAGE MEDIUM

(71) Applicant: Zhi Mei Kang Min (Zhuhai) Health Tech Co., Ltd., Zhuhai (CN)

(72) Inventors: Chao Wu, Princeton, NJ (US); Aizhen Li, Zhuhai (CN)

(21) Appl. No.: 17/906,639

(22) PCT Filed: Dec. 16, 2020
(86) PCT No.: PCT/CN2020/136749

§ 371 Date: Sep. 19, 2022

(30) Foreign Application Priority Data
Mar. 19, 2020 (CN) 202010194511.7

(51) Int. Cl. A61B 34/32; A61B 18/06; A61H 39/08; B25J 13/08

(52) U.S. Cl. CPC A61B 34/32; A61B 18/06; A61H 39/08; B25J 13/089; A61B 2562/0257; A61H 2201/1659; A61H 2201/5064; A61H 2201/5092

(57) ABSTRACT

A tool head posture adjustment method and apparatus, and a readable storage medium are disclosed. Distances to laser points are measured by means of laser distance sensors, and the laser points can construct at least one plane, thereby determining each plane corresponding to a site to be checked; and a comprehensive normal vector of each plane is calculated, and posture parameters of a tool head of a robot are calculated by using a posture expression, thereby adjusting the tool head posture.

### CROSS-REFERENCE TO RELATED APPLICATIONS

This application is a national stage filing under 35 U.S.C. § 371 of international application number PCT/CN2020/136749, filed 16 Dec., 2020, which claims priority to Chinese patent application No. 2020101945117 filed 19 Mar. 2020. The contents of these applications are incorporated herein by reference in their entirety.

### TECHNICAL FIELD

The disclosure relates to the technical field of robots, in particular to a tool head posture adjustment method and apparatus, and a readable storage medium.

### BACKGROUND

With the development of robot technology, the performance and safety of robots have been greatly improved, and the emergence of robots capable of human-machine cooperation has remarkably made human-robot cooperation be practical. With the rapid development of modern industry in the last decade, a widespread use of robots has also made the cost of robots approach the affordable level of consumers in the last two years. As a kind of robots, the personal care robot includes a tool head with a care function (massage, moxibustion, making up, beautifying, etc.) mounted at an end of the robot arm, and the tool head is in direct contact with the human body to be cared or maintains a specific distance and posture to carry out care work.

At present, the solutions to determine the posture (position and attitude) of a care robot during work mainly include a machine vision solution, a contacting multi-axis force sensor solution, and a radar (ultrasonic or laser, etc.) sensor. The above-mentioned solutions are all imperfect. For example, the robots involved in the machine vision solution are expensive. According to the robot vision solution, a special light source is required to irradiate, scanning time and coordinate generation time are long, real-time performance is poor, the camera with a large volume is not conducive to being integrated in the tool head, and the private parts of clients are exposed. The robots involved in the contacting multi-axis force sensor solution are expensive, which is only adaptable to a scenario where a tool head is in contact with a human body, and has no prospective guidance on the tool head posture. The robots involved in the radar solution are expensive, poor in accuracy, large in blind areas, susceptible to interference, and have a potential risk to human health from the emission of long-term radar waves. Therefore, how to provide a tool head posture determination solution which is low in cost, high in precision, strong in real-time performance and accurate in posture determination has become a problem to be solved.

The above-mentioned content is only used to assist in understanding the technical solutions of the disclosure, and does not represent an admission that the above-mentioned content is the prior art.

### SUMMARY

A main object of the disclosure is to provide a tool head posture determination solution which is low in cost, high in precision, strong in real-time performance and accurate in posture determination.

In order to achieve the above object, the disclosure provides a tool head posture adjustment method, including:
controlling laser points of laser distance sensors to irradiate onto a site to be checked in a way that the laser points are not on a straight line, where there are more than two of the laser distance sensors;
acquiring a distance measured by each of the laser distance sensors, an initial coordinate of each of the laser distance sensors and a laser direction of each of the laser distance sensors, where the distance is a distance between each laser distance sensor and the corresponding laser point;
calculating a laser point coordinate of each of the laser points on the site to be checked based on the distance, the initial coordinate and the laser direction, and calculating a comprehensive plane normal vector of a plane determined by the laser point coordinates, where the comprehensive plane normal vector is obtained from a plane normal vector of the plane determined by the laser point coordinates; and
calculating posture parameters of a tool head posture of the robot to be adjusted based on a preset posture expression, the distances and the comprehensive plane normal vector, and controlling a tool head of the robot to be adjusted to a posture to be adjusted based on the posture parameters.

Optionally, if there are more than three of the laser distance sensors, calculating a comprehensive normal vector of a plane determined by the laser point coordinates includes:
calculating plane normal vectors of corresponding planes determined by the laser point coordinates; and
calculating a weighted average value of the plane normal vectors using a weighted average algorithm, and taking the weighted average value as the comprehensive plane normal vector.

Optionally, the posture expression includes an Euler angle, a quaternion, or a rotation matrix expression.

Optionally, there are three of the laser distance sensors, and laser beams of the laser distance sensors are parallel to one another.

Optionally, calculating a laser point coordinate of each of the laser points on the site to be checked based on the distance, the initial coordinate and the laser direction includes:

assuming unit vectors of the laser directions to be Rₐ(rₓ₁, r_{y1}, r_{z1}), R_{b}(rₓ₂, r_{y2}, r_{z2}) and R_{c}(rₓ₃, r_{y3}, r_{z3}) respectively, the coordinates of the laser distance sensors in a tool head coordinate system to be Pₐ(x₁, y₁, z₁), Pb(x₂, y₂, z₂) and Pc(x₃, y₃, z₃), the distances to be d₁, d₂ and d₃, and the coordinates of the laser points to be Uₐ, U_{b} and U_{c}, then

$${U}_{a . x} = {x}_{1} + {r}_{x 1} * {d}_{1} , {U}_{a . y} = {y}_{1} + {r}_{y 1} * {d}_{1} , {U}_{a . z} = {z}_{1} + {r}_{z 1} * {d}_{1} ;$$

$${U}_{b . x} = {x}_{2} + {r}_{x 2} * {d}_{2} , {U}_{b . y} = {y}_{2} + {r}_{y 2} * {d}_{2} , {U}_{b . z} = {z}_{2} + {r}_{z 2} * {d}_{2} ;$$

$${U}_{c . x} = {x}_{3} + {r}_{x 3} * {d}_{3} , {U}_{c . y} = {y}_{3} + {r}_{y 3} * {d}_{3} , {U}_{c . z} = {z}_{3} + {r}_{z 3} * {d}_{3} ;$$

to obtain the coordinates of the laser points Uₐ(U_{a.x}, U_{a.y}, U_{a.z}), U_{b}(U_{b.x}, U_{b.y}, U_{b.z}) and U_{c}(U_{c.x}, U_{c.y}, U_{c.z}).

Optionally, calculating posture parameters of a tool head posture of the robot to be adjusted based on a preset posture expression, the distances and the comprehensive plane normal vector includes:
assuming the obtained comprehensive plane normal vector to be V(vₓ, v_{y}, v_{z}), with the convention that v_{z}>0, where if v_{z}<0, the comprehensive plane normal vector is multiplied by −1;
presetting a formula M(α, β, γ)*[0, 0, 1]^{T}=Vₙₒᵣₘ^{T}, where M(α, β, γ) is a rotation matrix in an Euler angle rotation formula, where Vₙₒᵣₘ represents the comprehensive plane normal vector with modulo being 1, and α, β and γ respectively represent Euler angles; and
obtaining the posture parameters based on the preset formula, the distances and the Euler angle rotation formula.

Optionally, obtaining the posture parameters based on the preset formula, the distances and the Euler angle rotation formula includes:
assuming rₓ to be equal to 0, and calculating r_{y} and r_{z} based on the preset formula and the Euler angle rotation formula, where rₓ, r_{y} and r_{z} are Euler angles α, β and γ, respectively;
calculating a comprehensive distance using a preset algorithm based on the distances, which is assumed to be dₐᵥₑᵣ;
adding dₐᵥₑᵣ to an origin of the tool head coordinate system in z direction so that the origin is set at the site to be checked;
acquiring posture parameters of the origin with respect to a base coordinate system of a manipulator base when the origin is provided at the site to be checked, where the posture parameters are assumed to be x₀, y₀, z₀, rₓ₀, r_{y0} and r_{z0}; and
assuming Euler angle parameters of the tool head posture to be adjusted to be Rx, Ry and R_{z}, then
Rₓ=rₓ₀, R_{y}=r_{y0}+r_{y}, and R_{z}=r_{z0}+r_{z}, then the posture parameters of the tool head posture to be adjusted being x₀, y₀, z₀, Rₓ, R_{y} and R_{z}.

Optionally, the preset algorithm includes an average algorithm and a weighted average algorithm.

Further, in order to achieve the above object, the disclosure further provides a tool head posture adjustment apparatus, including: a memory, a processor, and a tool head posture adjustment program stored on the memory and executable by the processor, when executed by the processor, the tool head posture adjustment program implements the method as described above.

Further, in order to achieve above object, the disclosure further provides a readable storage medium, storing thereon a tool head posture adjustment program which, when executed by a processor, implements the tool head posture adjustment method as described above.

The disclosure provides a tool head posture adjustment method and apparatus, and a readable storage medium, which can accurately determine the tool head posture of a robot, thereby improving the working efficiency of the robot. According to the disclosure, the laser distance sensors are low in cost, small in size and high in accuracy, can be well integrated with the tool head of a robot, so as to facilitate the determination of the tool head posture, can adjust the positions of the laser distance sensors in real time. The disclosure can adapt to a scenario where a tool head is in contact with a human body or a scenario where the tool head is kept at a certain distance from the human body.

### BRIEF DESCRIPTION OF THE DRAWINGS

FIG. 1 is a schematic flowchart of a tool head posture adjustment method according to a first embodiment of the disclosure;

FIG. 2 is a schematic view of the moxibustion instrument robot performing acupuncture on a site to be checked:

FIG. 3 is a schematic view of three laser distance sensors on the tool head in the first embodiment.

The objects, features and advantages of the disclosure will be further described with reference to the accompanying drawings in conjunction with the embodiments.

### DETAILED DESCRIPTION

In order to make the purpose, technical solutions and advantages of the disclosure clearer, the disclosure will be described in further detail below in conjunction with the accompanying drawings and embodiments. It should be understood that the specific embodiments described herein are only used to explain the disclosure and not intended to limit the disclosure.

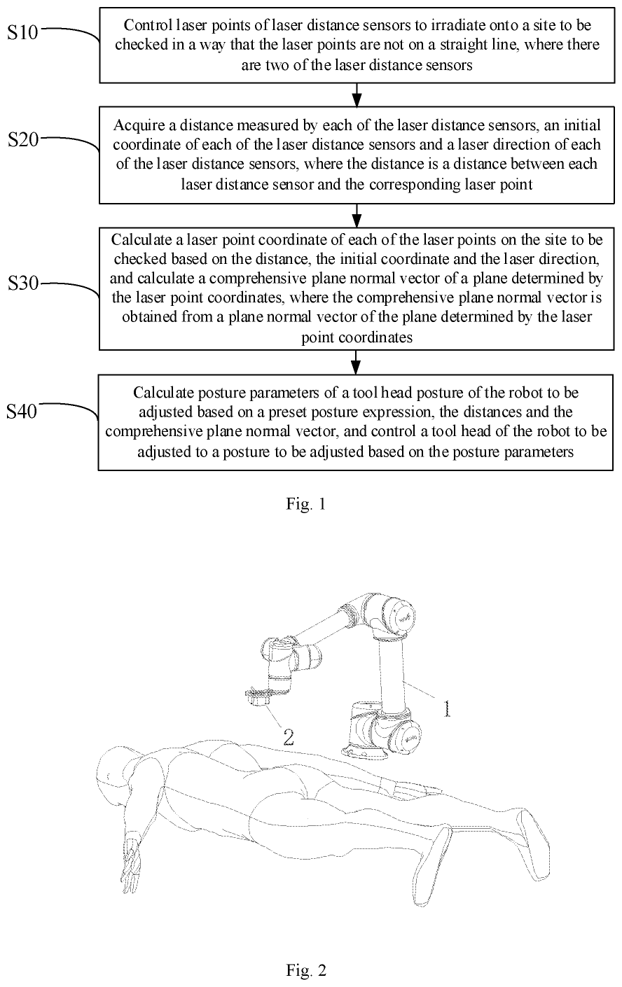

The disclosure further provides a tool head posture adjustment method. Referring to FIG. 1, which is a schematic flowchart of a tool head posture adjustment method according to a first embodiment of the disclosure.

In the present embodiment, the tool head posture adjustment method is executed by a tool head posture adjustment system, which includes a tool head posture adjustment apparatus, and the tool head posture adjustment apparatus may be a robot device, such as a moxibustion instrument and a massage instrument, but may also be a terminal device, such as a PC and a palmtop computer. According to the disclosure, distances to laser points are measured by means of laser distance sensors, and the various laser points can construct at least one plane, thereby determining each plane corresponding to a site to be checked; and a comprehensive normal vector of the planes is calculated, and posture parameters of a tool head of a robot are calculated by using a posture expression, thereby adjusting the tool head posture. According to the tool head posture adjustment method and apparatus, posture parameters of a tool head posture to be adjusted can be accurately determined. The present embodiment takes the moxibustion instrument as an example for illustration. The tool head posture adjustment method includes the following steps.

At S10, laser points of laser distance sensors are controlled to irradiate onto a site to be checked in a way that the laser points are not on a straight line, where the number of the laser distance sensors is more than 2.

In the present embodiment, referring to FIG. 2, which is a schematic view of the moxibustion instrument robot carrying out acupuncture at a site to be checked, where the site to be checked includes a body site of a patient to be acupunctured, and the laser distance sensors may be mounted on a tool head of the moxibustion instrument robot, for example, three or more laser distance sensors 2 are mounted on a face of the robot tool head having a fixed linkage relationship, and certainly, can also be mounted on other parts of the robot. A manipulator 1 is used to adjust the tool head posture. When the site to be checked enters the range of the distance from the laser distance sensors 2, laser beams of the respective laser distance sensors 2 irradiate onto the site to be checked, forming laser points at the site to be checked, the number of the laser distance sensors 2 is more than or equal to 3. Since the laser points must be able to construct a plane, the laser points cannot be on the same straight line. Preferably, the laser beams of the laser distance sensor 2 are prevented from converging at a point, and the laser beams are parallel to each other and never have an intersection point, so that the algorithm can be simplified when calculating the coordinates of the laser points, thereby improving the efficiency of data processing and further improving the efficiency of adjusting the tool head posture.

At S20, a distance measured by each of the laser distance sensors, an initial coordinate of each of the laser distance sensors and a laser direction of each of the laser distance sensors are acquired, where the distance is a distance between each laser distance sensor and the corresponding laser point.

In the present embodiment, the laser distance sensors 2 can measure a distance to a laser point, set a tool head coordinate system, and acquire initial coordinates of the laser distance sensors 2 in the tool head coordinate system and laser directions of the laser distance sensors 2, where the laser directions of the laser distance sensors 2 can be represented by a unit vector. At S30, a laser point coordinate of each of the laser points on the site to be checked is calculated based on the distance, the initial coordinate and the laser direction, and a comprehensive plane normal vector of a plane determined by the laser point coordinates are calculated, where the comprehensive plane normal vector is obtained from a plane normal vector of a plane determined by the laser point coordinates.

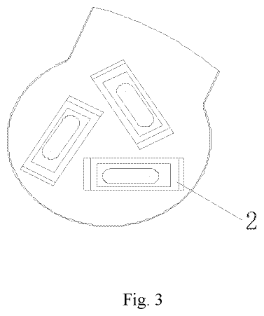

In the present embodiment, referring to FIG. 3, which is a schematic diagram of three laser distance sensors 2 on the tool head, if there are three laser distance sensors 2, the comprehensive plane normal vector is a plane normal vector of the plane where the three laser points are located, where the plane normal vector may be obtained by vector cross multiplication. If there are more than three laser distance sensors 2, the comprehensive normal vector may be a normal vector obtained using a weighted average algorithm or other algorithms. For example, if the laser points can construct four planes, the normal vectors of the four planes are respectively calculated by vector cross multiplication, and then a weighted average value of the four normal vectors is calculated by a weighted average algorithm, thereby obtaining a comprehensive normal vector.

If the number of the laser distance sensors 2 is 3, the laser beams of the laser distance sensors 2 are parallel to each other, unit vectors of the respective laser directions are set as Rₐ(rₓ₁, r_{y1}, r_{z1}), R_{b}(rₓ₂, r_{y2}, r_{z2}) and R_{c}(rₓ₃, r_{y3}, r_{z3}), respectively, the coordinates of the laser distance sensors in the tool head coordinate system are set as Pₐ(x₁, y₁, z₁), P_{b}(x₂, y₂, z₂) and P_{c}(x₃, y₃, z₃), the respective distances are set as d₁, d₂ and d₃, and the coordinates of the laser points are set as Uₐ, U_{b} and U_{c}, then

$${U}_{a . x} = {x}_{1} + {r}_{x 1} * {d}_{1} , {U}_{a . y} = {y}_{1} + {r}_{y 1} * {d}_{1} , {U}_{a . z} = {z}_{1} + {r}_{z 1} * {d}_{1} ;$$

$${U}_{b . x} = {x}_{2} + {r}_{x 2} * {d}_{2} , {U}_{b . y} = {y}_{2} + {r}_{y 2} * {d}_{2} , {U}_{b . z} = {z}_{2} + {r}_{z 2} * {d}_{2} ;$$

$${U}_{c . x} = {x}_{3} + {r}_{x 3} * {d}_{3} , {U}_{c . y} = {y}_{3} + {r}_{y 3} * {d}_{3} , {U}_{c . z} = {z}_{3} + {r}_{z 3} * {d}_{3} ;$$

that is, the coordinates Uₐ(U_{a.x}, U_{a.y}, U_{a.z}), U_{b}(U_{b.x}, U_{b.y}, U_{b.z}) and U_{c}(U_{c.x}, U_{c.y}, U_{c.z}) of the laser points are obtained, and the plane normal vector of the three laser points may be calculated by vector cross multiplication.

At S40, posture parameters of a tool head posture of the robot to be adjusted are calculated based on a preset posture expression, the distances and the comprehensive plane normal vectors, and the tool head of the robot to be adjusted is controlled to the posture to be adjusted based on the posture parameters.

In the present embodiment, the preset posture expression includes expressions such as an Euler angle, a quaternion or a rotation matrix, and the posture parameters of the posture of the robot to be adjusted are obtained by using the posture expression according to the comprehensive plane normal vector and the distances.

The Euler angle rotation formula is used to calculate the posture parameters of the posture of the robot to be adjusted hereinafter.

The obtained comprehensive plane normal vector is assumed to be V(vₓ, v_{y}, v_{z}), with the convention that v_{z}>0, where if v_{z}<0, the comprehensive plane normal vector is multiplied by −1, which is to calculate the unit normal vector of the comprehensive plane normal vector, so as to determine the direction of the comprehensive normal vector.

The Euler angle rotation formula is:

$$M ( \text{} α , \text{} β , \text{} γ ) = [ \begin{matrix}\text{} cos α os α c - cos β sin α in α s & - cos β cos γ os γ s - cos α o s α s & sin α sin β \\ cos γ os γ s + cos α os αβ sin λ & cos α os αβ cos γ - sin α in α s & - cos α o s α β \\ sin β sin λ & cos γ os γβ & cos β\end{matrix} ]$$

Assuming the preset formula M(α, β, γ)*[0, 0, 1]^{T}=Vₙₒᵣₘ^{T}, where M(α, β, γ) is a rotation matrix in the Euler angle rotation formula, α, β, and γ respectively represent Euler angles, Vₙₒᵣₘ represents the comprehensive plane normal vector with modulo being 1, and α, β and γ respectively represent Euler angles.

Let rₓ to be equal to 0, r_{y} and r_{z} are obtained based on the preset formula and the Euler angle rotation formula, where rₓ, r_{y} and r_{z} are Euler angles α, β and γ, respectively, a comprehensive distance is calculated using a preset algorithm based on a distance, and is set as dₐᵥₑᵣ: dₐᵥₑᵣ is added to an origin of a tool head coordinate system in a z direction so that the origin is set at a site to be checked: then posture parameters of the origin with respect to a base coordinate system of a manipulator base are acquired when the origin is provided at the site to be checked, and the posture parameters are set as x₀, y₀, z₀, rₓ₀, r_{y0} and r_{z0}; and the Euler angle parameters of the tool head posture to be adjusted are set as Rₓ, R_{y} and R_{z}, then Rₓ=rₓ₀, R_{y}=r_{y0}+r_{y}, and R_{z} r_{z0}+r_{z}, then the posture parameters of the tool head posture to be adjusted are x₀, y₀, z₀, Rₓ, R_{y} and R_{z}, where the preset algorithm includes an average algorithm and a weighted average algorithm, for example, an average value of d₁, d₂ and ds is calculated using the average algorithm to obtain dₐᵥₑᵣ.

The tool head posture is adjusted according to the posture parameters of the posture to be adjusted, so that the robot can accurately perform acupuncture and moxibustion care on the site to be checked.

The present embodiment provides a tool head posture adjustment method, including: controlling laser points of laser distance sensors to irradiate onto a site to be checked in a way that the laser points are not on a straight line, where the number of the laser distance sensors is more than 2: acquiring a distance measured by each of the laser distance sensors, an initial coordinate of each of the laser distance sensors and a laser direction of each of the laser distance sensors, where the distance is a distance between each laser distance sensor and the corresponding laser point; calculating a laser point coordinate of each of the laser points on the site to be checked based on the distance, the initial coordinate and the laser direction, and calculating a comprehensive plane normal vector of a plane determined by the laser point coordinates, where the comprehensive plane normal vector is obtained from a plane normal vector of the plane determined by the laser point coordinates; and calculating posture parameters of a tool head posture of the robot to be adjusted based on a preset posture expression, the distances and the comprehensive plane normal vector, and controlling the tool head of the robot to be adjusted to the posture to be adjusted based on the posture parameters. In this way, the posture parameters of the tool head posture to be adjusted can be accurately determined, thereby improving the operation efficiency of the moxibustion instrument. In addition, the laser distance sensors of the disclosure are low in cost, high in precision and strong in real-time performance when the robot works, and can adapt to a scenario where a tool head is in contact with a human body or a scenario where the tool head is kept at a certain distance from the human body.

In addition, the embodiments of the disclosure also provide a readable storage medium.

The readable storage medium of the disclosure stores thereon a tool head posture adjustment program, which, when executed by a processor, implements the following steps:
controlling laser points of laser distance sensors to irradiate onto a site to be checked in a way that the laser points are not on a straight line, where the number of the laser distance sensors is more than 2;
acquiring a distance measured by each of the laser distance sensors, an initial coordinate of each of the laser distance sensors and a laser direction of each of the laser distance sensors, where the distance is a distance between each laser distance sensor and the corresponding laser point;
calculating a laser point coordinate of each of the laser points on the site to be checked based on the distance, the initial coordinate and the laser direction, and calculating a comprehensive plane normal vector of a plane determined by the laser point coordinates, where the comprehensive plane normal vector is obtained from a plane normal vector of the plane determined by the laser point coordinates; and
calculating posture parameters of a tool head posture of the robot to be adjusted based on a preset posture expression, the distances and the comprehensive plane normal vector, and controlling a tool head of the robot to be adjusted to a posture to be adjusted based on the posture parameters.

Further, if there are more than three of the laser distance sensors, calculating a comprehensive normal vector of a plane determined by the laser point coordinates includes:
calculating plane normal vectors of corresponding planes determined by the laser point coordinates; and
calculating a weighted average value of the plane normal vectors using a weighted average algorithm, and taking the weighted average value as the comprehensive plane normal vector.

Further, the posture expression includes an Euler angle, a quaternion, or a rotation matrix expression.

Further, there are three of the laser distance sensors, and laser beams of the laser distance sensors are parallel to one another.

Further, calculating a laser point coordinate of each of the laser points on the site to be checked based on the distance, the initial coordinate and the laser direction includes:
assuming unit vectors of the laser directions to be Rₐ(rₓ₁, r_{y1}, r_{z1}), R_{b}(rₓ₂, r_{y2}, r_{z2}) and R_{c}(rₓ₃, r_{y3}, r_{z3}) respectively, the coordinates of the laser distance sensors in a tool head coordinate system to be Pₐ(x₁, y₁, z₁), P_{b}(x₂, y₂, z₂) and P_{c}(x₃, y₃, z₃), the distances to be d₁, d₂ and d₃, and the coordinates of the laser points to be Uₐ, U_{b} and U_{c}, then

$${U}_{a . x} = {x}_{1} + {r}_{x 1} * {d}_{1} , {U}_{a . y} = {y}_{1} + {r}_{y 1} * {d}_{1} , {U}_{a . z} = {z}_{1} + {r}_{z 1} * {d}_{1} ;$$

$${U}_{b . x} = {x}_{2} + {r}_{x 2} * {d}_{2} , {U}_{b . y} = {y}_{2} + {r}_{y 2} * {d}_{2} , {U}_{b . z} = {z}_{2} + {r}_{z 2} * {d}_{2} ;$$

$${U}_{c . x} = {x}_{3} + {r}_{x 3} * {d}_{3} , {U}_{c . y} = {y}_{3} + {r}_{y 3} * {d}_{3} , {U}_{c . z} = {z}_{3} + {r}_{z 3} * {d}_{3} ;$$

to obtain the coordinates of the laser points, where Pₐ, P_{b} and P_{c} are respectively the coordinates Uₐ(U_{a.x}, U_{a.y}, U_{a.z}), U_{b}(U_{b.x}, U_{b.y}, U_{b.z}) and U_{c}(U_{c.x}, U_{c.y}, U_{c.z}) of the laser distance sensors.

Further, calculating posture parameters of a tool head posture of the robot to be adjusted based on a preset posture expression, the distances and the comprehensive plane normal vector includes:
assuming the obtained comprehensive plane normal vector to be V(vₓ, v_{y}, v_{z}), with the convention that v_{z}>0, where if v_{z}<0, the comprehensive plane normal vector is multiplied by −1;
presetting a formula M(α, β, γ)*[0, 0, 1]^{T}=Vₙₒᵣₘ^{T}, where M(α, β, γ) is a rotation matrix in an Euler angle rotation formula; and
obtaining the posture parameters based on the preset formula, the distances and the Euler angle rotation formula, where Vₙₒᵣₘ represents the comprehensive plane normal vector with modulo being 1, and α, β and γ respectively represent Euler angles.

Further, obtaining the posture parameters based on the preset formula, the distances and the Euler angle rotation formula includes:
assuming rₓ to be equal to 0, and calculating r_{y} and r_{z} based on the preset formula and the Euler angle rotation formula, where rₓ, r_{y} and r_{z} are Euler angles α, β and γ, respectively;
calculating a comprehensive distance using a preset algorithm based on the distances, which is assumed to be dₐᵥₑᵣ;
adding dₐᵥₑᵣ to an origin of the tool head coordinate system in z direction so that the origin is set at the site to be checked;
acquiring posture parameters of the origin with respect to a base coordinate system of a manipulator base when the origin is provided at the site to be checked, where the posture parameters are assumed to be x₀, y₀, z₀, rₓ₀, r_{y0} and r_{z0}; and
assuming Euler angle parameters of the tool head posture to be adjusted to be Rₓ, R_{y} and R_{z}, then
Rₓ=rₓ₀, R_{y}=r_{y0}+r_{y}, and R_{z}=r_{z0}+r_{z}, then the posture parameters of the tool head posture to be adjusted being x₀, y₀, z₀, Rₓ, R_{y} and R_{z}.

Further, the preset algorithm includes an average algorithm and a weighted average algorithm.

It should be noted that, as used herein, the terms “comprising”, “including” or any other variation thereof are intended to encompass non-exclusive inclusion, such that a process, method, article or system including a series of elements includes not only those elements, but also other elements that are not explicitly listed, or also includes elements inherent to such a process, method, article, or system. Without further limitation, an element defined by the statement “includes a . . . ” does not preclude the existence of additional identical elements in the process, method, article or system that includes the element.

The above-mentioned serial numbers of the embodiments of the disclosure are merely for the purpose of description and do not represent the advantages and disadvantages of the embodiments.

From the description of the embodiments given above, it will be clear to a person of ordinary skill in the art that the method of the embodiments described above can be implemented by means of software plus a necessary general hardware platform, and certainly also by means of hardware, but in many cases the former is a better way. Based on such understanding, the technical solutions of the disclosure essentially or the part that contributes to the prior art can be reflected in the form of software products, and the computer software products are stored in a storage medium (such as an ROM/RAM, a magnetic diskette and an optical disk) as described above, and include several instructions for making a terminal device (which may be a mobile phone, a computer, a server, an air conditioner, or a network device, etc.) to execute the method according to various embodiments of the disclosure.

The above are only preferred embodiments of the disclosure, and do not therefore limit the patent scope of the disclosure. All equivalent structures or equivalent process changes made by using the description of the disclosure and the accompanying drawings, or directly or indirectly used in other related technical fields, are equally included in the scope of patent protection of the disclosure.

1. A tool head posture adjustment method, applicable to a robot, comprising:
controlling laser points of laser distance sensors to irradiate onto a site to be checked in a way that the laser points are not on a straight line, where there are more than two of the laser distance sensors;
acquiring a distance measured by each of the laser distance sensors, an initial coordinate of each of the laser distance sensors and a laser direction of each of the laser distance sensors, where the distance is a distance between each laser distance sensor and the corresponding laser point;
calculating a laser point coordinate of each of the laser points on the site to be checked based on the distance, the initial coordinate and the laser direction, and calculating a comprehensive plane normal vector of a plane determined by the laser point coordinates, where the comprehensive plane normal vector is obtained from a plane normal vector of the plane determined by the laser point coordinates; and
calculating posture parameters of a tool head posture of the robot to be adjusted based on a preset posture expression, the distances and the comprehensive plane normal vector, and controlling a tool head of the robot to be adjusted to a posture to be adjusted based on the posture parameters.

2. The tool head posture adjustment method according to claim 1, wherein if there are more than three of the laser distance sensors, calculating a comprehensive normal vector of a plane determined by the laser point coordinates comprises:
calculating plane normal vectors of corresponding planes determined by the laser point coordinates; and
calculating a weighted average value of the plane normal vectors using a weighted average algorithm, and taking the weighted average value as the comprehensive plane normal vector.

3. The tool head posture adjustment method according to claim 1, wherein the posture expression comprises an Euler angle, a quaternion, or a rotation matrix expression.

4. The tool head posture adjustment method according to claim 1, wherein
there are three of the laser distance sensors, and laser beams of the laser distance sensors are parallel to one another.

5. The tool head posture adjustment method according to claim 4, wherein
calculating a laser point coordinate of each of the laser points on the site to be checked based on the distance, the initial coordinate and the laser direction comprises:
assuming unit vectors of the laser directions to be Ra(rx1, ry1, rz1), Rb(rx2, ry2, rz2) and Rc(rx3, ry3, rz3) respectively, the coordinates of the laser distance sensors in a tool head coordinate system to be Pa(x1, y1, z1), Pb(x2, y2, z2) and Pc(x3, y3, z3), the distances to be d1, d2 and d3, and the coordinates of the laser points to be Ua, Ub and Uc, then
$$Ua . x = x 1 + rx 1 * d 1 , Ua . y = y 1 + ry 1 * d 1 , Ua . z = z 1 + rz 1 * d 1 ;$$
$$Ub . x = x 2 + rx 2 * d 2 , Ub . y = y 2 + ry 2 * d 2 , Ub . z = z 2 + rz 2 * d 2 ;$$
$$Uc . x = x 3 + rx 3 * d 3 , Uc . y = y 3 + ry 3 * d 3 , Uc . z = z 3 + rz 3 * d 3 ;$$
to obtain the coordinates of the laser points Ua(Ua.x, Ua.y, Ua.z), Ub(Ub.x, Ub.y, Ub.z) and Uc(Uc.x, Uc.y, Uc.z).

6. The tool head posture adjustment method according to claim 3, wherein calculating posture parameters of a tool head posture of the robot to be adjusted based on a preset posture expression, the distances and the comprehensive plane normal vector comprises:
assuming the obtained comprehensive plane normal vector to be V(vx, vy, vz), with the convention that vz>0, where if vz<0, the comprehensive plane normal vector is multiplied by −1;
presetting a formula M(α, β, γ)*[0, 0, 1]T=VnormT, where M(α, β, γ) is a rotation matrix in an Euler angle rotation formula, where Vnorm represents the comprehensive plane normal vector with modulo being 1, and α, β and γ respectively represent Euler angles; and
obtaining the posture parameters based on the preset formula, the distances and the Euler angle rotation formula.

7. The tool head posture adjustment method according to claim 6, wherein obtaining the posture parameters based on the preset formula, the distances and the Euler angle rotation formula comprises:
assuming rx to be equal to 0, and calculating ry and rz based on the preset formula and the Euler angle rotation formula, where rx, ry and rz are Euler angles α, β and γ, respectively;
calculating a comprehensive distance using a preset algorithm based on the distances, which is assumed to be daver;
adding daver to an origin of the tool head coordinate system in z direction so that the origin is set at the site to be checked;
acquiring posture parameters of the origin with respect to a base coordinate system of a manipulator base when the origin is provided at the site to be checked, where the posture parameters are assumed to be x0, y0, z0, rx0, ry0 and rz0; and
assuming Euler angle parameters of the tool head posture to be adjusted to be Rx, Ry and Rz, then
Rx=rx0, Ry=ry0+ry, and Rz=rz0+rz, then the posture parameters of the tool head posture to be adjusted being x0, y0, z0, Rx, Ry and Rz.

8. The tool head posture adjustment tool head posture adjustment method according to claim 7, wherein the preset algorithm comprises an average algorithm and a weighted average algorithm.

9. A tool head posture adjustment apparatus, comprising: a memory, a processor, and a tool head posture adjustment program stored on the memory and executable by the processor, when executed by the processor, the tool head posture adjustment program implements a tool head posture adjustment method, applicable to a robot, comprising:
controlling laser points of laser distance sensors to irradiate onto a site to be checked in a way that the laser points are not on a straight line, where there are more than two of the laser distance sensors;
acquiring a distance measured by each of the laser distance sensors, an initial coordinate of each of the laser distance sensors and a laser direction of each of the laser distance sensors, where the distance is a distance between each laser distance sensor and the corresponding laser point;
calculating a laser point coordinate of each of the laser points on the site to be checked based on the distance, the initial coordinate and the laser direction, and calculating a comprehensive plane normal vector of a plane determined by the laser point coordinates, where the comprehensive plane normal vector is obtained from a plane normal vector of the plane determined by the laser point coordinates; and
calculating posture parameters of a tool head posture of the robot to be adjusted based on a preset posture expression, the distances and the comprehensive plane normal vector, and controlling a tool head of the robot to be adjusted to a posture to be adjusted based on the posture parameters.

10. A non-transitory computer readable storage medium, storing thereon a tool head posture adjustment program, which, when executed by a processor, implements a tool head posture adjustment method, applicable to a robot, comprising:
controlling laser points of laser distance sensors to irradiate onto a site to be checked in a way that the laser points are not on a straight line, where there are more than two of the laser distance sensors;
acquiring a distance measured by each of the laser distance sensors, an initial coordinate of each of the laser distance sensors and a laser direction of each of the laser distance sensors, where the distance is a distance between each laser distance sensor and the corresponding laser point;
calculating a laser point coordinate of each of the laser points on the site to be checked based on the distance, the initial coordinate and the laser direction, and calculating a comprehensive plane normal vector of a plane determined by the laser point coordinates, where the comprehensive plane normal vector is obtained from a plane normal vector of the plane determined by the laser point coordinates; and
calculating posture parameters of a tool head posture of the robot to be adjusted based on a preset posture expression, the distances and the comprehensive plane normal vector, and controlling a tool head of the robot to be adjusted to a posture to be adjusted based on the posture parameters.
